(19) United States
(12) Patent Application Publication
(10) Pub. No.: US 2004/0170989 A1
(43) Pub. Date: Sep. 2, 2004

(54) Cellular gene targets for controlling cell growth

(76) Inventors: Xin Katherine Ye, Fremont, CA (US); Robert Stull, Alameda, CA (US); Sergey A. Axenovich, San Jose, CA (US); Patrick Michael O'Connor, San Diego, CA (US)

Correspondence Address: SHERIDAN ROSS PC, 1560 BROADWAY, SUITE 1200, DENVER, CO, 80202

(21) Appl. No.: 10/441,925

(22) Filed: May 19, 2003

Related U.S. Application Data
(60) Provisional application No. 60/381,619, filed on May 17, 2002.

(51) Int. Cl. C12Q 1/68; G01N 33/53; G01N 33/574

(52) U.S. Cl. 435/6; 435/7.23

(57) ABSTRACT

A method of identifying a compound that induces apoptosis in a cell is disclosed. The method includes contacting the cell with a putative apoptosis-inducing compound and determining whether the compound inhibits a target selected from the group consisting of APLP2, ENSA, FBN2, FIBL-6, PRNP, STRN4, XPR1, SLC5A6, SLC31A1, GFER, and PCTK1. Also disclosed are methods for inducing apoptosis in a cell by inhibiting one of the targets. The invention further includes methods for the diagnosis of a tumor that include determining the level of one of the targets as a marker in a patient sample, the level of the marker being indicative of the presence of tumor cells.

### REFERENCE TO RELATED APPLICATIONS

This application claims priority under 35 U.S.C. §119(e) from U.S. Provisional Patent Application No. 60/381,619, filed on May 17, 2002, which is incorporated herein by reference in its entirety.

### FIELD OF THE INVENTION

The present invention relates to methods for inducing apoptosis in cells by inhibiting targets involved in the suppression of apoptosis, and to identifying compounds useful in such methods.

### BACKGROUND OF THE INVENTION

The p53 tumor suppressor protein is an essential component in the regulation of the cell cycle, senescence, and programmed cell death (apoptosis). This protein regulates transcription of many genes in response to DNA damage and various transforming stimuli. The functional inactivation of p53 can occur through the action of viral oncoproteins, or through over-expression of the hdm2 (human) or mdm2 (murine) oncogene protein. Additional tumor suppressors, such as the p14^{ARF} product of the INK4a gene, regulate the functional activity of p53. In the case of p14^{ARF}, the suppressor interacts with hdm2 and thereby prevents the mentioned oncoprotein from inhibiting p53. An alternative translation product of the INK4a locus, p16INK4a, a cyclin-dependent kinase inhibitor, also contributes to normal growth control through its regulation of the Rb pathway.

When regulation of the cell cycle, senescence, and apoptosis is not functioning properly, uncontrolled cell growth and tumor formation occurs. Because of the complicated regulation of these cell functions, there are many potential points in a variety of regulatory pathways of a cell for intervention. By inhibiting the expression of genes important to cell growth and to suppression of apoptosis or the proteins encoded by them, it is possible to induce control cell growth and apoptosis in a cell, thereby preventing tumor formation. Once such genes or proteins are identified as targets, assays can be conducted for drug discovery to find inhibitors suitable for use as therapeutic agents. In addition, such genes or proteins are useful as markers of tumor formation.

There is an ongoing need to identify new targets and develop new assays for the identification of therapeutic compounds useful in the control of cell growth and tumor formation.

### SUMMARY OF THE INVENTION

This invention provides methods for identifying compounds that induce apoptosis by inhibiting cellular genes or gene products involved in the control of cell growth. The present invention also includes a method for inducing apoptosis in a cell by inhibiting such a target gene or gene product by contacting cells susceptible to uncontrolled growth with an inhibitory compound in an amount sufficient to inhibit said biochemical activity or expression. More particularly, targets of the present invention include APLP2, ENSA, FBN2, FIBL-6, PRNP, STRN4, XPR1, SLC5A6, SLC31A1, GFER, and PCTK1.

In one embodiment, the present invention is a method of identifying a compound that induces apoptosis in a cell that includes contacting the cell with a putative apoptosis-inducing compound and determining whether the compound inhibits a target selected from the group consisting of APLP2, ENSA, FBN2, FIBL-6, PRNP, STRN4, XPR1, SLC5A6, SLC31A1, GFER, and PCTK1. The target can have been validated as being involved in tumor cell growth, such as by a process of inhibiting the target in a cell by a method selected from gene knock-out, anti-sense oligonucleotide expression, use of RNAi molecules and GSE expression or assaying the cell for the ability of the cell to grow. The cell can be a tumor cell line. The step of determining can be selected from assaying for reduced expression of the target and assaying for reduced activity of the target. The expression of the target can be measured by polymerase chain reaction or by using an antibody that specifically recognizes the target. The activity of the target can be measured by measuring the amount of a product generated in a biochemical reaction mediated by the target or by measuring the amount of a substrate consumed in a biochemical reaction mediated by the target. The inhibitor can be identified by determining the three-dimensional structure of the target or by determining the three-dimensional structure of an inhibitor by using computer software capable of modeling the interaction of the target and putative test compounds.

Another embodiment of the present invention is a method for inducing apoptosis in a cell by inhibiting a target selected from APLP2, ENSA, FBN2, FIBL-6, PRNP, STRN4, XPR1, SLC5A6, SLC31A1, GFER, and PCTK1.

A further embodiment of the present invention is a method for the diagnosis of a tumor that includes determining the level of a marker selected from APLP2, ENSA, FBN2, FIBL-6, PRNP, STRN4, XPR1, SLC5A6, SLC31A1, GFER, and PCTK1 in a patient sample. In this method, the level of the marker is indicative of the presence of tumor cells. In this method, the level of the marker can be determined by identifying the marker as a cell surface molecule in tissue or by detecting the marker in soluble form in a bodily fluid, such as serum, that can be immobilized. The marker level can be determined by contacting a patient sample with an antibody, or a fragment thereof, that binds specifically to the marker and determining whether the anti-marker antibody or fragment has bound to the marker. The marker level can be determined by using a first monoclonal antibody that binds specifically to the marker and a second antibody that binds to the first antibody. This method can be used to determine the prognosis for cancer in the patient or to determine the susceptibility of the patient to a therapeutic treatment.

### DESCRIPTION OF THE DRAWINGS

FIG. 1a is a schematic illustration of the V99 vector.

FIG. 1b is a schematic illustration of the construction of the V99 vector.

FIG. 1c is a flowchart of the random fragment library construction process for the POLA1U library.

FIG. 1d is a flowchart of the mRNA normalization process.

FIG. 1e illustrates a gel of quality control transcripts used in the mRNA normalization process.

FIG. 1f illustrates a gel of results in the mRNA normalization process.

FIG. 1g illustrates the directional fragmentation strategy for the random fragment library construction.

FIG. 1h is a flow chart of the functional screening process of the invention.

FIG. 2 is a schematic illustration of the construction of the V85 vector.

### DETAILED DESCRIPTION OF THE PREFERRED EMBODIMENTS

The invention includes methods for identifying protective compounds that control cell growth and induce apoptosis by using human cellular genes that encode products necessary for protecting cells from apoptosis as targets in the design of therapeutic agents. The invention further includes compounds for use in the treatment or prevention of tumor growth. Such compounds include chemical compounds and biological compounds. Chemical compounds or biological compounds include any chemical or biological compound that disrupts or inhibits one or more biological functions required for controlling cell growth. Preferred chemical compounds include small molecule inhibitor or substrate compounds, such as products of chemical combinatorial libraries. Preferred biological compounds include peptides, anti-sense molecules and antibodies.

Targets of the present invention have been identified as corresponding to genetic suppressor elements (GSEs) that control cell growth. The GSX™ System technology allows rapid screening for the inhibitors of gene function in the form of GSEs, which are gene fragments, that, when expressed in cells, act as genetic inhibitors of the corresponding intact gene in those cells. A GSE can exert its effect through either an antisense, or a dominant negative peptide mechanism. GSEs are selected from libraries of DNA fragments, generated by random breakage of sets of test genes, cloned in a retroviral or other expression vector. The random fragment library clones are introduced into a population of test cells at approximately one test fragment per cell. Cells with a desired new phenotype, resulting from the expression of a GSE, are isolated on the basis of any selectable parameter. The GSEs are recovered from the selected cells and characterized by DNA sequence analysis and further functional assays.

As used herein, the term “isolated nucleic acid molecule” refers to a nucleic acid molecule that has been removed from its natural milieu (i.e., a molecule that has been subject to human manipulation) and can include DNA, RNA, or derivatives of either DNA or RNA. An isolated nucleic acid molecule can be isolated from its natural source or can be produced using recombinant DNA technology (e.g., polymerase chain reaction amplification) or chemical synthesis. Isolated nucleic acid molecules include natural nucleic acid molecules and homologs thereof, including, but not limited to, natural allelic variants and modified nucleic acid molecules in which nucleotides have been inserted, deleted, substituted, or inverted in such a manner that such modifications do not substantially interfere with the nucleic acid molecule's ability to control cell growth.

It should also be appreciated that reference to an isolated nucleic acid molecule does not necessarily reflect the extent of purity of the nucleic acid molecule. Nucleic acid molecules can be isolated and obtained in substantial purity, generally as other than an intact chromosome. Usually, the nucleic acid molecule will be obtained substantially free of other nucleic acid sequences, generally being at least about 50%, and usually at least about 90% pure. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably.

According to the invention, reference to an “isolated nucleic acid molecule” refers to a nucleic acid molecule that is the size of or smaller than a gene. Thus, an isolated nucleic acid molecule does not encompass isolated genomic DNA or an isolated chromosome. The term isolated nucleic acid molecule does not connote any specific minimum length. As used herein, the term “gene” has the meaning that is well known in the art, that is, a nucleic acid sequence that includes the translated sequences that code for a protein (“exons”) and the untranslated intervening sequences (“introns”), and any regulatory elements ordinarily necessary to translate the protein.

“Hybridization” has the meaning that is well known in the art, that is, the formation of a duplex structure by two single-stranded nucleic acids due to complementary base pairing. Hybridization can occur between exactly complementary nucleic acid strands or between nucleic acid strands that contain some regions of mismatch.

The present invention is based, in part, on the Applicants' isolation of certain GSEs from human cells that prevent cell growth, and that such nucleic acid molecules correspond to fragments of certain human cellular genes. In that regard, any cellular phenotype or protein associated with cell growth can be used to select for such nucleic acid molecules. An example of such a protein is p53.

GSEs having the ability to control cell growth can be functional in the sense orientation (and encode a peptide thereby), and can be functional in the antisense orientation (and encode antisense RNAs thereby). These GSEs are believed to down-regulate the corresponding cellular gene from which they were derived by different mechanisms. Such a corresponding cellular gene is referred to herein as a “target gene” and its product is referred to as a “target product.” As used herein, the term “target” alone refers collectively to a target gene and its corresponding target product. Sense-oriented GSEs exert their effects as transdominant mutants or RNA decoys. Transdominant mutants are expressed proteins or peptides that competitively inhibit the normal function of a wild-type protein in a dominant fashion. RNA decoys are protein binding sites that titrate out these wild-type proteins. Anti-sense oriented GSEs exert their effects as antisense RNA molecules, i.e., nucleic acid molecules complementary to the mRNA of the target gene. These nucleic acid molecules bind to mRNA and block the translation of the mRNA. In addition, some antisense nucleic acid molecules can act directly at the DNA level to inhibit transcription.

Specific target genes of the present invention are shown below in the Examples section in Table 1. The products of such target genes are target products of the present invention. Methods of the present invention for identifying therapeutic compounds by identifying an inhibitor of a target in the human host cell include identifying an inhibitor of: a target gene from Table 1, as well as target products encoded by any of the foregoing. More specifically, the targets of the present invention include APLP2, ENSA, FBN2, FIBL-6, PRNP, STRN4, XPR1, SLC5A6, SLC31A1, GFER, and PCTK1.

In one embodiment of the invention, the down-regulation of the concentration or activity of a target gene or product by an inhibitor (including a GSE) depletes a cellular component required for protecting cells from apoptosis resulting in control of cell growth. In another embodiment of the invention, the down-regulation of the concentration or activity of one target gene or product by an inhibitor (including a GSE) depletes a cellular component that interacts with another human cellular gene or gene product required for protecting cells from apoptosis resulting in control of cell growth. In a preferred embodiment of the invention, the two human cellular genes are members of the same biological pathway and one human cellular gene or gene product regulates the expression or activity of the other human cellular gene or gene product. In another preferred embodiment of the invention, the two human cellular genes are members of the same biological pathway and the substrate of a polypeptide encoded by one human cellular gene is a product of a biochemical reaction mediated by the polypeptide encoded by the other human cellular gene. In still another preferred embodiment of the invention, the two human cellular genes are members of the same biological pathway and the product of a polypeptide encoded by one human cellular gene is a substrate of a biochemical reaction mediated by the polypeptide encoded by the other human cellular gene. In another embodiment, the two human cellular genes encode polypeptides that are isozymes of each other. In a preferred embodiment, at least one of the human cellular genes encodes an enzyme.

It will be understood that this invention is not limited to the particular methodology, protocols, cell lines, animal species or genera, constructs, or reagents described herein, as such may vary. It is also to be understood that the terminology used herein is for the purpose of describing particular embodiments only, and is not intended to limit the scope of the invention that will be limited only by the appended claims. All technical and scientific terms used herein have the same meaning as commonly understood to one of ordinary skill in the art to which this invention belongs unless clearly indicated otherwise.

Target genes or proteins identified using GSEs can be further evaluated using a variety of methods to validate their involvement in cell growth, suppression of apoptosis and tumor formation. Such methods include methods that disrupt or “knock out” the expression of a target gene in a cell capable of apoptosis. Knock-out methods include somatic cell knock-outs and inhibitory RNA molecules including anti-sense oligonucleotides, siRNA molecules, RNAi molecules and RNA decoys. Target genes or proteins can also be evaluated by methods that include nucleic acid-based experiments such as Northern Blots, Real Time polymerase chain reaction or high density microarrays. Further evaluation can also be achieved using human/mouse xenograft models. For example, human tumor cells can be transfected with a GSE such that the GSE is expressed. Preferred tumor cells include HCT116 and MDA-MB-231. The transfected cells can then be implanted into mice, preferably nude mice. The growth of the tumor cells in the mouse can then be measured.

Once one or more members of a biological pathway are identified as required for cell growth, the present invention can include identifying additional members of a biological pathway that are also required for cell growth. Such subsequent identification is within the skill of one in the art. GSEs, and therefore preferred targets of the present invention, are identified by selecting cells that exhibit certain hallmarks of apoptosis upon expression of the GSEs. Isolated GSEs are further prioritized based on their specificity for a neoplastic transformation state, such as their activity in transformed and non-transformed cells, and based on the p53 pathway status in cells expressing the GSEs. For example, GSEs can be prioritized by determining if the GSEs have activity in a p53 dependent and/or independent manner. GSEs specific for the neoplastic transformation state are preferred for identifying targets for anti-cancer drugs.

Once a human cellular gene has been identified as a target for supporting cell growth, an assay can be used for screening and selecting a chemical compound or a biological compound having activity as an anti-tumor therapeutic based on the ability to down-regulate expression of the gene or inhibit activity of its gene product. Reference herein to inhibiting a target, refers to both inhibiting expression of a target gene and inhibiting the activity of its corresponding expression product. Such a compound is referred to herein as therapeutic compound. For example, a cell line that naturally expresses the gene of interest or has been transfected with the gene is incubated with various compounds. A reduction of the expression of the gene of interest or an inhibition of the activities of its encoded product may be used as to identify a therapeutic compound. Therapeutic compounds identified in this manner can then be re-tested in other assays to confirm their activities against apoptosis.

In one embodiment of the invention, inhibitors of cell growth are identified by exposing a mammalian cell to a test compound; measuring the expression of a human cellular gene or an activity of the polypeptide encoded by the human cellular gene in the mammalian cell; and selecting a compound that down-regulates the expression of the human cellular gene or interferes with the activities of its encoded product. A preferred mammalian cell to use in an assay is a mammalian cell that either naturally expresses the human cellular gene or has been transformed with a recombinant form of the human cellular gene. Methods to determine expression levels of a gene are well known in the art.

In a preferred embodiment, the expression of the human cellular gene is measured by the polymerase chain reaction. In another preferred embodiment, the expression of the human cellular gene is measured using an antibody that specifically recognizes the polypeptide encoded by the human cellular gene and is analyzed using methods such as immunoprecipitation, ELISAs, fluorescence activated cell sorting (FACS) and immunofluorescence microscopy. In another embodiment, the expression of the human cellular gene is measured using polyacrylamide gel analysis, chromatography or spectroscopy. In still another preferred embodiment, the activity of the polypeptide encoded by the human cellular gene is measured by measuring the amount of product. generated in a biochemical reaction mediated by the polypeptide encoded by the human cellular gene. In still another preferred embodiment, the activity of the polypeptide encoded by the human cellular gene is measured by measuring the amount of substrate generated in a biochemical reaction mediated by the polypeptide encoded by the target gene. In another embodiment of the invention, therapeutic compounds are selected by determining the three-dimensional structure of a human cellular gene product; and determining the three-dimensional structure of a therapeutic compound by rational drug design. Preferably, the structure of the therapeutic compound is determined using computer software capable of modeling the interaction of a therapeutic compound with the target gene. One of skill in the art can select the appropriate three-dimensional structure, therapeutic compound, and analytical software based on the identity of the target gene.

In still another embodiment of the invention, inhibitors of cell growth are identified by exposing a polypeptide encoded by a target gene to a test compound; measuring the binding of the test compound to the polypeptide; and selecting a compound that binds to the polypeptide at a desired concentration, affinity, or avidity. In a preferred embodiment, the assay is performed under conditions conducive to promoting the interaction or binding of the compound to the polypeptide. One of skill in the art can determine such conditions based on the polypeptide and the compound being used in the assay.

In still another embodiment of the invention, a therapeutic compound is identified by exposing an enzyme encoded by a target gene to a test compound; measuring the activity of the enzyme encoded by the target gene in the presence and absence of the compound; and selecting a compound that down-regulates or inhibits the activity of the enzyme encoded by the target gene. Methods to measure enzymatic activity are well known to those skilled in the art and are selected based on the identity of the enzyme being tested. For example, if the enzyme is a kinase, phosphorylation assays can be used.

In addition to methods for identifying and producing a biological compound that inhibits cell growth, the present invention includes methods known in the art that down-regulate expression or function of a target gene. For example, antisense RNA and DNA molecules may be used to directly block translation of mRNA encoded by these cellular genes by binding to targeted mRNA and preventing protein translation. Polydeoxyribonucleotides can form sequence-specific triple helices by hydrogen bonding to specific complementary sequences in duplexed DNA to effect specific down-regulation of target gene expression. Formation of specific triple helices may selectively inhibit the replication or expression of a target gene by prohibiting the specific binding of functional trans-acting factors.

Ribozymes are enzymatic RNA molecules capable of catalyzing the specific cleavage of RNA. Ribozyme action involves sequence specific hybridization of the ribozyme molecule to complementary target RNA, followed by endonucleolytic cleavage. Within the scope of the invention are ribozyme embodiments including engineered hammerhead motif ribozyme molecules that specifically and efficiently catalyze endonucleolytic cleavage of cellular RNA sequences. Antisense RNA molecules showing high-affinity binding to target sequences can also be used as ribozymes by addition of enzymatically active sequences known to those skilled in the art.

Polynucleotides to be used in triplex helix formation should be single-stranded and composed of deoxynucleotides. The base composition of these polynucleotides must be designed to promote triple helix formation via Hoogsteen base pairing rules, which generally require sizeable stretches of either purines or pyrimidines to be present on one strand of a duplex. Polynucleotide sequences may be pyrimidine-based, which will result in TAT and CGC triplets across the three associated strands of the resulting triple helix. The pyrimidine-rich polynucleotides provide base complementarity to a purine-rich region of a single strand of the duplex in a parallel orientation to that strand. In addition, polynucleotides may be chosen that are purine-rich, for example, containing a stretch of G residues. These polynucleotides will form a triple helix with a DNA duplex that is rich in GC pairs, in which the majority of the purine residues are located on a single strand of the targeted duplex, resulting in GGC triplets across the three strands in the triplex.

Alternatively, sequences that can be targeted for triple helix formation can be increased by creating a so-called “switchback” polynucleotide. Switchback polynucleotides are synthesized in an alternating 5′-3′, 3′-5′ manner, so that they base pair with first one strand of a duplex and then the other, eliminating the necessity for a sizeable stretch of either purines or pyrimidines to be present on one strand of a duplex.

Both antisense RNA and DNA molecules, and ribozymes of the invention may be prepared by any method known in the art. These include techniques for chemically synthesizing polynucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into host cells.

Various modifications to the nucleic acid molecules may be introduced as a means of increasing intracellular stability and half-life. Possible modifications include, but are not limited to, the addition of flanking sequences of ribonucleotides or deoxyribonucleotides to the 5′ or 3′ ends of the molecule or the use of phosphorothioate or 2′ O-methyl rather than phosphodiesterase linkages within the oligodeoxyribonucleotide backbone.

Preferably, methods used to identify therapeutic compounds are customized for each target gene or product. If the target product is an enzyme, then the enzyme will be expressed in cell culture and purified. The enzyme will then be screened in vitro against therapeutic compounds to look for inhibition of that enzymatic activity. If the target is a non-catalytic protein, then it will also be expressed and purified. Therapeutic compounds will then be tested for their ability to prevent, for example, the binding of a site-specific antibody or a target-specific ligand to the target product.

In a preferred embodiment, therapeutic compounds that bind to target products are identified, then those compounds can be further tested in biological assays that test for characteristics such as apoptosis, p53 status, tumor cell growth and any other customary measure of anti-cancer activity.

In one embodiment of the invention, a therapeutic compound is not toxic to a human host cell. In another embodiment, the therapeutic is cytostatic or cytotoxic.

In one embodiment of the invention, a pharmaceutical composition is prepared from a therapeutically-effective amount of a therapeutic compound of the invention and a pharmaceutically-acceptable carrier. Pharmaceutically-acceptable carriers are well known to those with skill in the art.

A further embodiment of the invention is a method for inducing apoptosis in a cell by inhibiting a target of the present invention, i.e., a target selected from the group consisting of APLP2, ENSA, FBN2, FIBL-6, PRNP, STRN4, XPR1, SLC5A6, SLC31A1, GFER, and PCTK1. For example, this method can be conducted in vivo by administering to an individual an inhibitory or therapeutic compound as generally discussed herein. In addition, the method can be conducted in vitro.

A further embodiment of the present invention is a method for the diagnosis of a tumor that includes determining the level of a marker in a patient sample, wherein the marker is selected from the group consisting of APLP2, ENSA, FBN2, FIBL-6, PRNP, STRN4, XPR1, SLC5A6, SLC31A1, GFER, and PCTK1. The level of the marker can be determined by conventional methods such as expression assays to determine the level of expression of the gene, by biochemical assays to determine the level of the gene product, or by immunoassays. If appropriate, the marker can be identified as a cell surface molecule in tissue or in a bodily fluid, such as serum. For example, a patient sample, which can be immobilized, can be contacted with an antibody, or an antibody fragment, that binds specifically to the marker and determining whether the anti-marker antibody or fragment thereof has bound to the marker. In a particular immunoassay, the marker level is determined using a first monoclonal antibody that binds specifically to the marker and a second antibody that binds to the first antibody.

If the level of the marker is greater than a normal level, the level of the marker is considered to be indicative of the presence of tumor cells. A normal level can be determined in a variety of ways. For example, if a patient history is known, a baseline level of the marker can be determined and higher levels will be indicative of tumor cells. Alternatively, a normal level can be based on the level for a healthy (i.e., without tumor) individual in a given population. That is, a normal level can be based on a population having similar characteristics (e.g., age, sex, race, medical history) as the patient in question.

This method of diagnosis can be used specifically to determine the prognosis for cancer in the patient or to determine the susceptibility of the patient to a therapeutic treatment.

The Examples, which follow, are illustrative of specific embodiments of the invention, and various uses thereof. They are set forth for explanatory purposes only, and are not to be taken as limiting the invention.

### EXAMPLE 1

The purpose of this experiment was to perform a nearly saturated genome wide GSE screen in a tumor cell line model for GSEs that inhibit target genes or products that protect cells against apoptosis.

1. V99 Vector Design and Construction

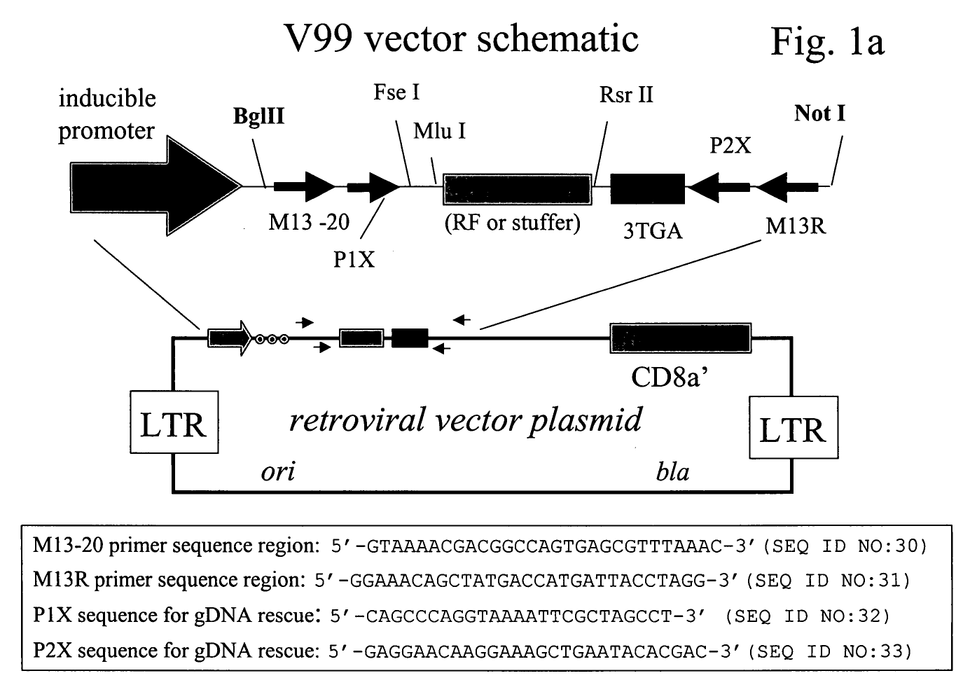

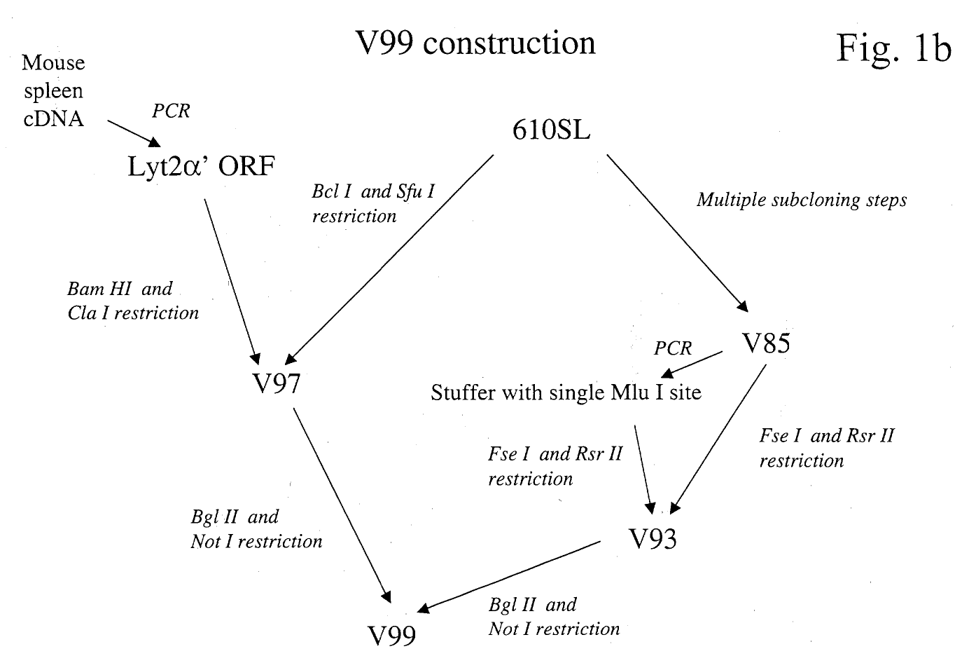

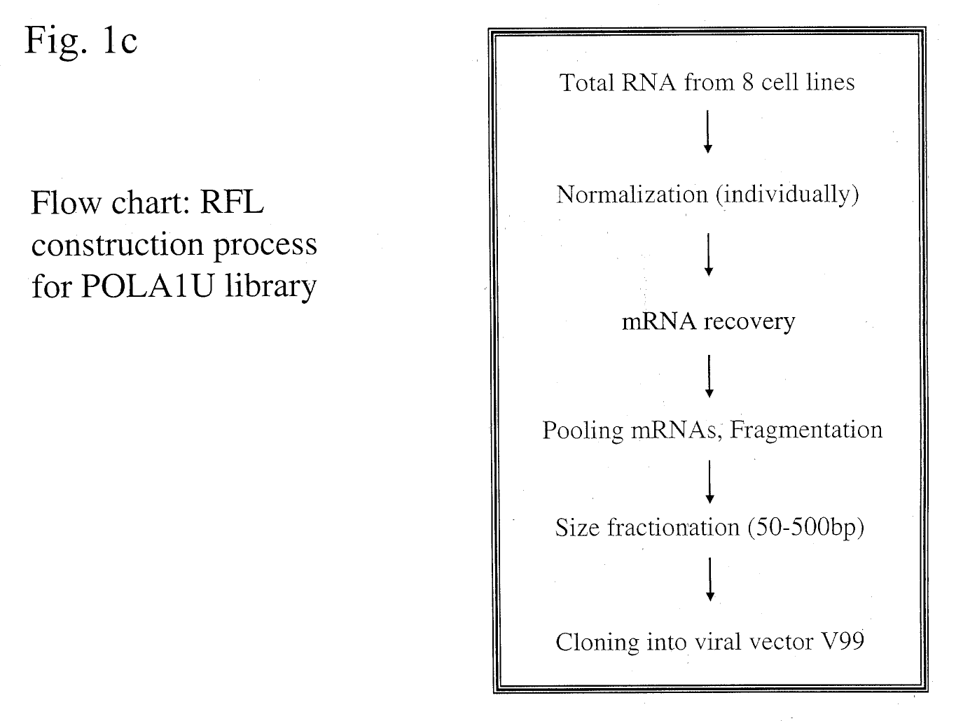

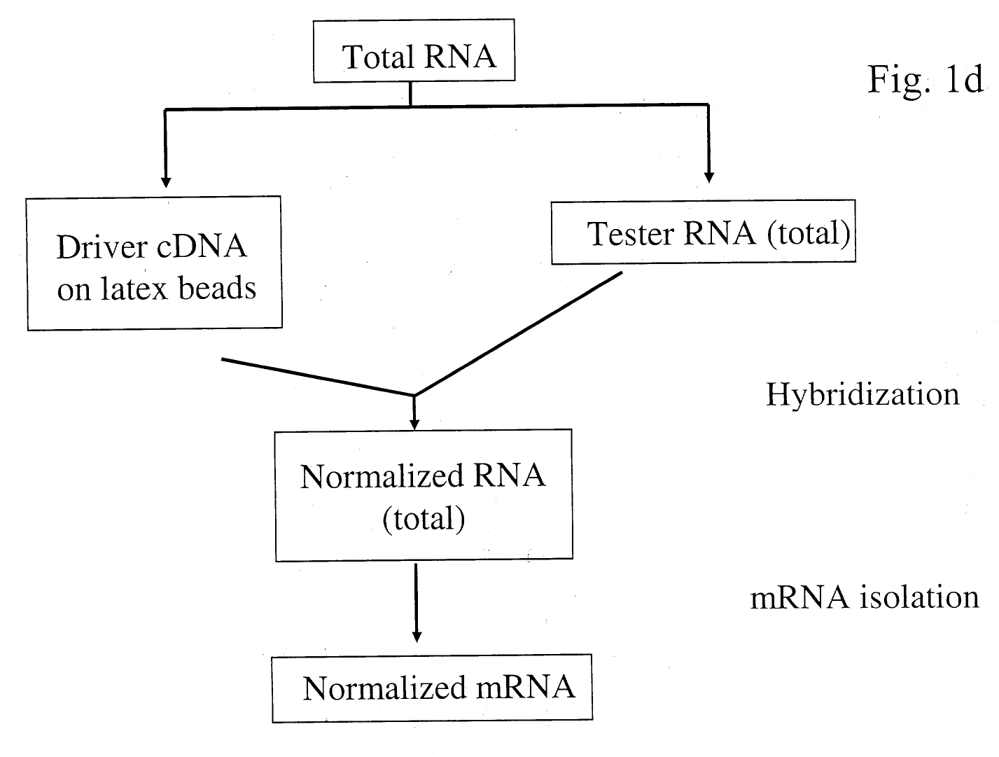

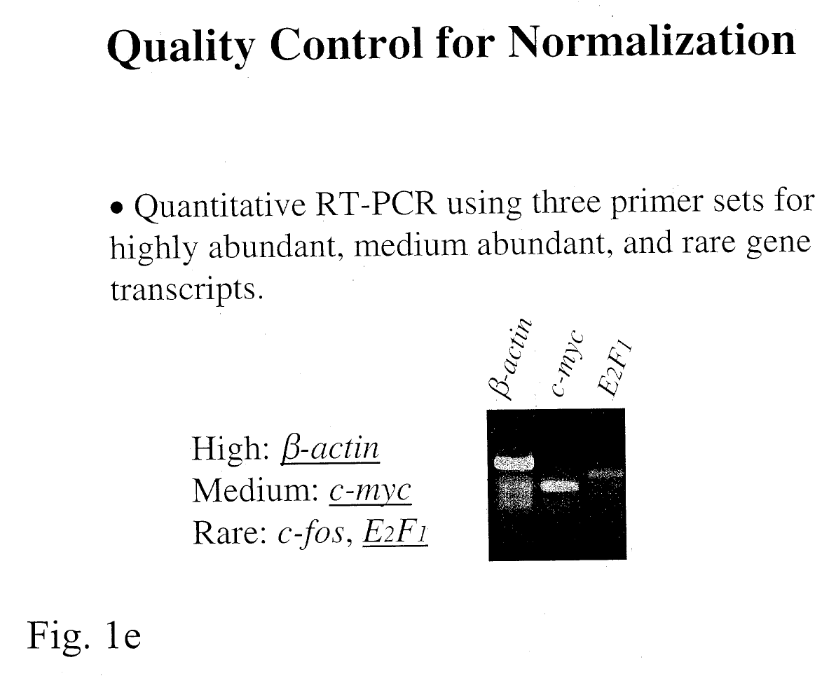

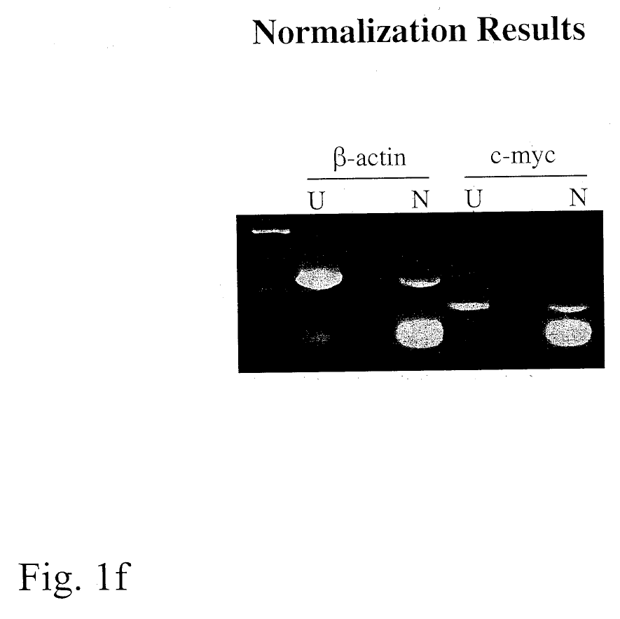

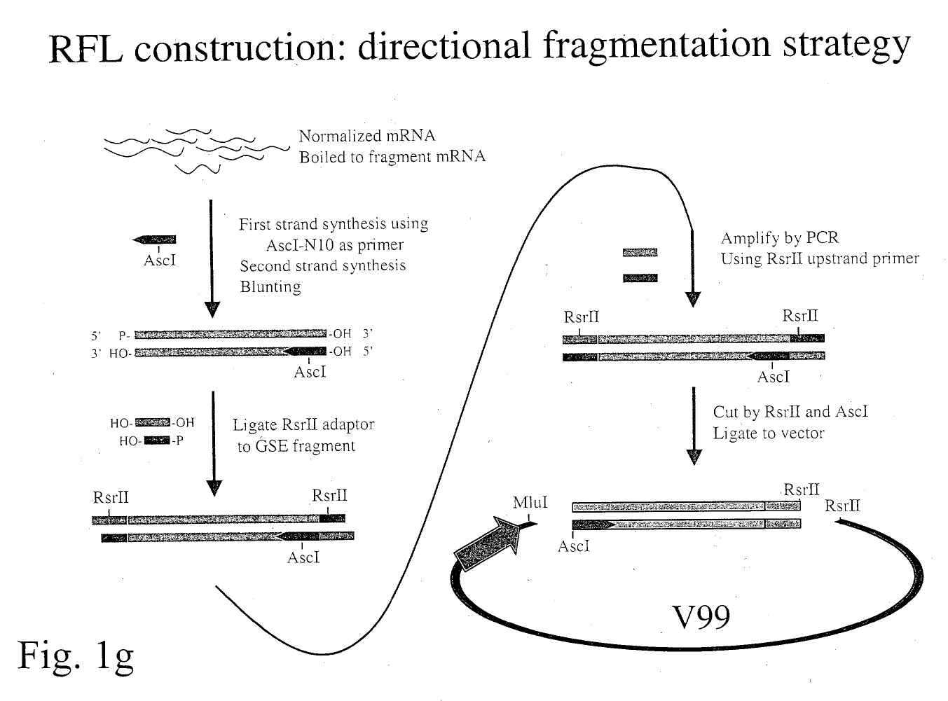

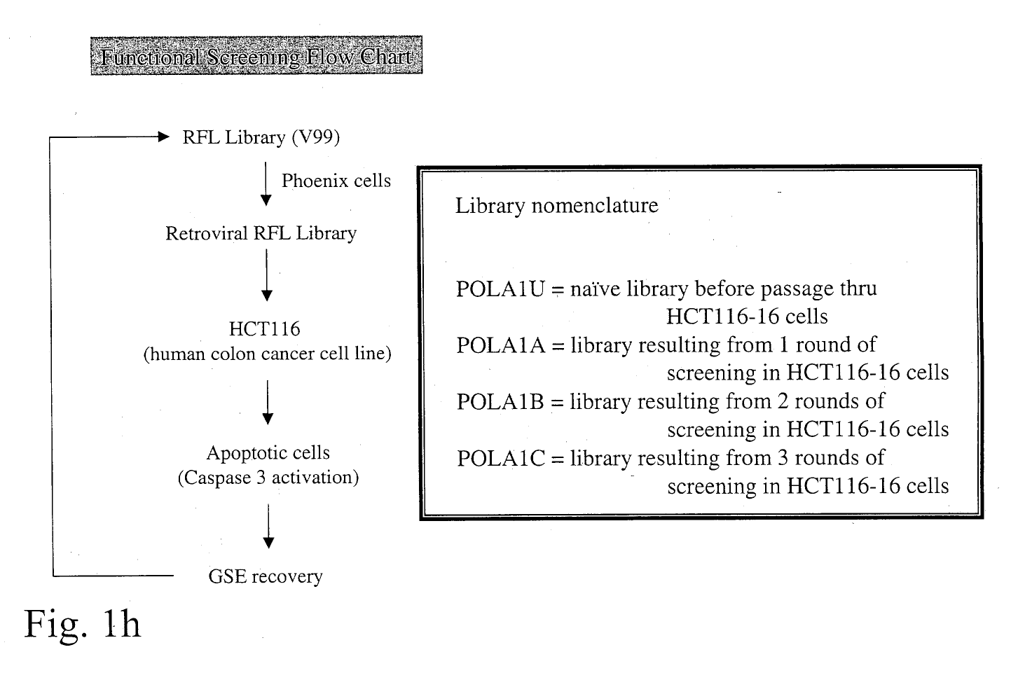

Vector V99 was created through modification of p610SL, a derivative of pLNCO₃ (B-D Chang and I. B. Roninson, Gene 183 (1996) 137-142.) A schematic of V99 is shown in FIG. 1. The region flanking the multiple cloning site (MCS) downstream of the inducible CMV promoter was re-engineered (1) to introduce restriction endonuclease sites for enzymes expected to occur with low frequency in the human genome [e.g., Fse I (1 per 170 kBp), Mlu I (1 per 300 kBp), and Rsr II (1 per 260 kBp)], (2) to introduce a short sequence of nucleic acid containing stop codons in all three DNA reading frames downstream of the MCS, (3) to introduce sequences complementary to well established DNA primers used for DNA sequencing (e.g., M13F-20 and M13R), to permit rapid and efficient sequencing of inserts cloned into the MCS, (4) to introduce sequences flanking the MCS, derived from the genome of Zea mays, and (5) to introduce into the MCS a “stuffer” fragment of about 2.2 kBp, which permits easy assessment of the completeness of vector digestion and selection of the appropriate backbone fragment during vector preparation.

A cDNA encoding the open reading frame of the murine Lyt-2-alpha′ gene was recovered from Marathon ready mouse spleen cDNA (Clontech) using PCR with the following conditions: 5 μL of Marathon ready cDNA, 5 μL 10× cDNA PCR buffer, 1 μL 10 mM dNTP mix, 1 μL Advantage 2 polymerase mix (Clontech, #8430-1), 1 μL of 10 μM upstream primer 5′-ACC ATG GCC TCA CCG TTG ACC CGC TTT-3′ (SEQ ID NO:1), 1 μL or 10 μM downstream primer 5′-CTA GCG GCT GTG GTA GCA GAT GAG A-3′ (SEQ ID NO:2), and 36 μL of water. Cycling parameters were: 94° C. for 3 min.; followed by 30 cycles of 94° C. for 30 sec., 55° C. for 30 sec., and 72° C. for 2 minutes; followed by 72° C. for 10 minutes; followed by a 4° C. soak. The resultant PCR product of 669 nucleotides was subcloned into a pCRII TOPO vector (InVitrogen). Several independent clones were sequenced to confirm no mutations were introduced in the Lyt-2-alpha′ ORF by the PCR. One pCRII-TOPO-Lyt-2-alpha′ clone was shown to be free of mutations, clone #2. DNA from clone #2 was subjected to a second round of PCR (Vt=50 μL) using the following conditions: 1 ng plasmid DNA, 5 μL 10× cDNA PCR buffer, 0.8 μL of 10 mM dNTP mix, 1 μL of Advantage 2 polymerase mix (Clontech, #8430-1), 2.5 μL of 10 μM upstream primer 5′-CTA CGG ATC CAC CAT GGC CTC ACC GTT GA-3′ (SEQ ID NO:3) and 2.5 μL of 10 μM downstream primer 5′-GTA CAT CGA TCT AGC GGC TGT GGT AGC AGA TGA GA-3′ (SEQ ID NO:4). These primers permitted recovery the ORF of the Lyt-2-alpha′ gene flanked by BamH I (upstream) and Cla I restriction endonuclease sites. Cycling parameters were: 94° C. for 3 min.; followed by 30 cycles of 94° C. for 30 sec., 55° C. for 30 sec., and 72° C. for 2 minutes; followed by 72° C. for 10 minutes; followed by a 4° C. soak. The resulting 689-bp PCR product was purified from surrounding proteins and salts using a Qiagen PCR clean up kit following manufacturer's instructions. The purified Clone #2 DNA digested with Bam HI restriction endonuclease (NEB, #R0136S). The digested product was purified using a Qiagen PCR clean up kit and the buffer was changed. The digested DNA was then further digested with Cla I restriction endonuclease (NEB, #R0197S). The doubly restricted Clone #2 DNA was then subcloned into the backbone fragment of the 610SL retroviral vector produced by double digestion of 610SL with Bcl I (NEB, #R0160S) and Sfu I (Roche, #1243497) restriction endonucleases. Sequencing of DNA harvested from several independent bacterial colonies that were produced from this subcloning step yielded a clone that showed no mutations in the Lyt-2-alpha′ ORF. This clone was named V97.

The modifications to the MCS regions of vector 610SL were created by sequential cloning of various double stranded oligonucleotides containing the desired sequences into several precursor plasmids. Sequences designed to be located 5′ to the Fse I GSE cloning site in V99, e.g., M13F-20 primer site, primer site for P1X, were created by subcloning annealed oligonucleotides 5′-AGC TGT AAA ACG ACG GCC AGT GAG CGT TTA AAC GAA TTC CAG ACT AGT GGC CGG CCG TGC A-3′ (SEQ ID NO:5) and 5′ CGG CCG GCC ACT AGT CTG GAA TTC GTT TAA ACG CTC ACT GGC CGT CGT TTT AC-3′ (SEQ ID NO:6) into the vector pEFGP-1 (ClonTech) between the HinD III and Pst I sites, to create pEGFP5′. The duplex produced by annealing primers 5′-AAT TCT GCA GCC CAG GTA AAA TTC GCT AGC CT-3′ (SEQ ID NO:7) and 5′-CTA GAG GCT AGC GAA TTT TAC CTG GGC TGC AG-3′ (SEQ ID NO:8), which contains the priming site for P1X sequence, was subcloned between the Eco RI and Spe I sites of pEGFP5′ to yield pEGFP54. The modified 5′ region of the MCS was recovered from plasmid pEFGP54 as a Bgl II-Not I flanked fragment, and subcloned between the Bgl II and Not I sites of p610SL, to yield p610-E54P1. Sequences designed to be located 3′ to the Rsr II GSE cloning site in V99, e.g., 3 frame stop cassette, primer P2X, M13R sequencing primer, were created by subcloning of annealed oligonucleotides 5′-CGG TCC GTG AGT GAG TGA GGC GCG CC G GAT CCT AAC CTA GGT AAT CAT GGT CAT AGC TGT TTC CTG CAG GGC-3′ (SEQ ID NO:9) and 5′-GGC CGC CCT GCA GGA AAC AGC TAT GAC CAT GAT TAC CTA GGT TAG GAT CCG GCG CGC CTC ACT CAC TCA CGG ACC GTG CA-3′ (SEQ ID NO:10) into the vector pBlueScript II (Stratagene) between the Pst I and Not I sites, to create plasmid pBS3.3′. The duplex produced by annealing primers 5′-GAT CCC GGG TCG TGT ATT CAG CTT TCC TTG TTC CT-3′ (SEQ ID NO:11) and 5′-CTA GAG GAA CAA GGA AAG CTG AAT ACA CGA CCC GG-3′ (SEQ ID NO:12), which contains the priming site for P2X sequence, was subcloned between the BamH I and Avr II sites of pBS3.3′ to yield pBS3.3′P12.

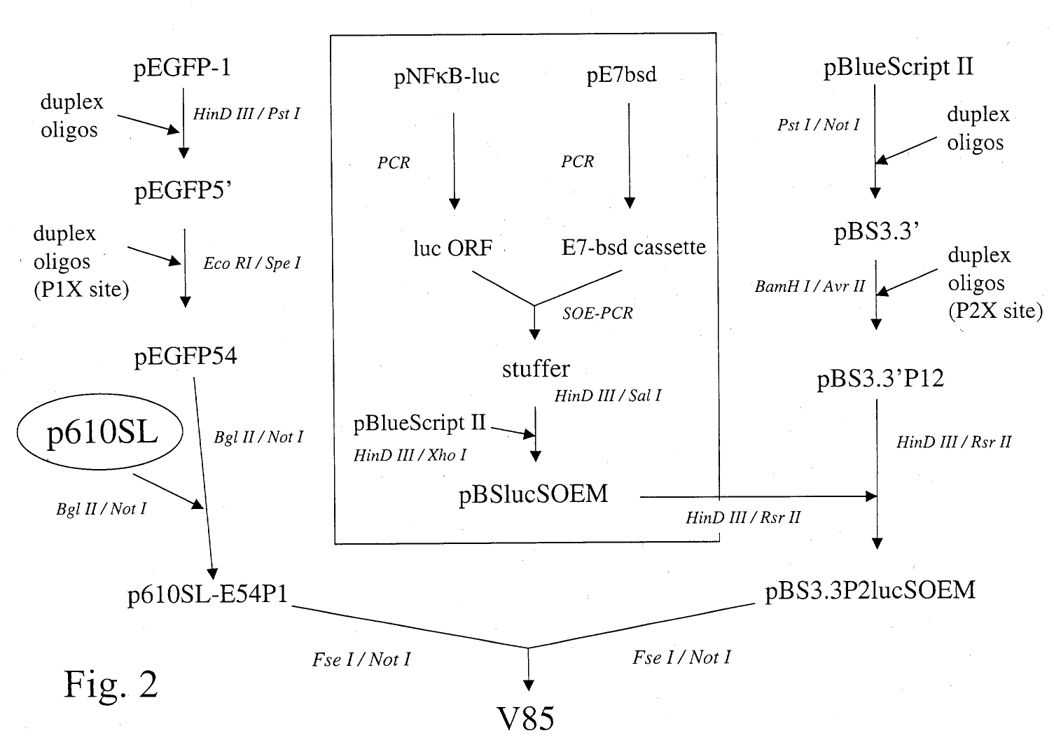

The stuffer fragment for V99 was designed to contain a luciferase ORF joined to a prokaryotic blasticidin S transferase (bsd) expression cassette, in order to yield a 2.2 kBp DNA fragment. The luciferase ORF and was created by PCR using the following primers 5′-CAT CAA GCT TGG CCG GCC ACG CGT GTT GGT AAA ATG GAA GAC G-3′ (SEQ ID NO:13) and 5′-CAC GTG GAT ATC TTA CAA TTT GGA CTT TCC GCC CT-3′ (SEQ ID NO:14) to amplify the luciferase ORF from the plasmid pNFκB-luc (Strategene, #219078), while the bsd expression cassette was created by PCR using the primers 5′-TTG TAA GAT ATC CAC GTG TTG ACA ATT AAT C-3′ (SEQ ID NO:15) and 5′-CAT CAG ATC TGT CGA CCG GAC CGA CGC GTC CAC GAA GTG CTT AGC-3′ (SEQ ID NO:16) to amplify the E7-blasticindin S transferase open reading frame cassette from plasmid EM7-bsd. (InVitrogen, #V511-20). Both reactions were performed using the following cycling parameters: 95° C. for 3 min; followed by 30 cycles of 94° C. for 30 sec., 60° C. for 30 sec., 72° C. for 2 min.; followed by 72° C. for 10 min.; followed by a soak at 4° C. PCR products of the desired size were purified by agarose gel electrophoresis followed by recovery of the DNA from the gel using the Qiagen Gel Extraction kit according to manufacturer's instructions. The luciferase ORF and the bsd expression cassette were spliced together to generate a 2.2 kBp stuffer fragment using splice overlap extension PCR (Horton, R. M., Hunt, H. D., Ho, S. N., Pullen, J. K. and Pease, L. R. (1989)). Engineering hybrid genes without the use of restriction enzymes: gene splicing by overlap extension. Gene 77, 61-68) and the primers 5′-CAT CAA GCT TGG CCG GCC ACG CGT GTT GGT AAA ATG GAA GAC G-3′ (SEQ ID NO:17) and 5′-CAT CAG ATC TGT CGA CCG GAC CGA CGC GTC CAC GAA GTG CTT AGC-3′ (SEQ ID NO:18). The resultant SOE PCR product was purified away from the proteins, primers, and salts using a Qiagen PCR clean up kit and following manufacturer's instructions. The product was digested with HinD III and Sal I restriction endonucleases, the restricted product was purified by agarose gel electrophoresis followed by recovery of the DNA from the gel using the Qiagen Gel Extraction kit according to manufacturer's instructions, and the purified DNA was subcloned between the HinD III (NEB, #R0104S) and Xho I (NEB, #R0146S) sites of plasmid pBluescript to yield pBSlucSOEM. Plasmid pBSlucSOEM was sequenced to confirm it was free of unwanted mutations, and the stuffer fragment recovered from the pBSIucSOEM as a HinD III and Rsr II fragment, which was purified by agarose gel electrophoresis, followed by recovery of the DNA from the gel using the Qiagen Gel Extraction kit according to manufacturer's instructions and subcloning of the fragment into the HinD III and Rsr II sites of plasmid pBS3.3′P12 to yield plasmid pBS33P2lucSOEM. Plasmid V85 was then constructed by recovering from plasmid pBS33P2lucORFM the luciferase-E7-bsd stuffer fragment along with the 3′ flanking sequences as an Fse I—Not I flanked 2.2 kBp DNA product, which was purified by agarose gel electrophoresis followed by recovery of the DNA from the gel using the Qiagen Gel Extraction kit according to manufacturer's instructions. This 2.2 kBp DNA was subcloned between the Fse I and Not I site of plasmid p610-E54P1, to yield vector V85. A schematic drawing of the construction of V85 is shown in FIG. 2.

The downstream Mlu I site was removed from V85 by PCR amplification of the stuffer fragment of V85 using 1 ng of V85 template DNA and 2.5 μL of primer 5′-CAT CAA GCT TGG CCG GCC ACG CGT GTT GGT AAA ATG GAA GAC G-3′ (SEQ ID NO:19) and 2.5 μL of primer 5′-CAT CGT CGA CCG GAC CGC CAC GAA GTG CTT AGC-3′ (SEQ ID NO:20) in a standard 50 μL PCR reaction using Taq DNA polymerase (Roche, 1146165). Cycling parameters were: 94° C. for 3 min.; followed by 30 cycles of 94° C. for 20 sec., 55° C. for 20 sec., and 72° C. for 3 minutes; followed by 72° C. for 10 minutes; followed by a 4° C. soak. The resultant 2.2 kBp PCR product was purified from the proteins and salt using a Qiagen PCR clean up kit following manufacturer's instructions. The PCR product was digested with Fse I and Rsr II endonucleases, purified by agarose gel electrophoresis, and recovered from the gel using a Qiagen Gel Extraction kit according to manufacturer's instructions. The restricted and purified fragment was then subcloned into the Fse I and Rsr II sites of V85 to yield V93. The MCS GSE cassette was recovered from V93 as a 2.2 kBp DNA fragment by digestion of V93 with Bgl II (NEB, #R0144S) and Not I (NEB, #R0189S) restriction endonucleases. Vector V99 was created by subcloning this 2.2 kBp DNA fragment from V93 into the Bgl II and Not I sites on the V97 backbone.

2. Random Fragment Library Construction

For construction of the starting POLA1U library, V99 vector described above was restricted at 37° C. for 3 hours, using Mlu I (NEB, #R0198S) and Rsr II restriction endonucleases. For construction of all other selected libraries, e.g., POLA1A, POLA1B, POLA1C, V99 vector DNA was restricted at 37° C. for 3 hours, using Fse I and Rsr II restriction endonucleases. The vector DNA was purified from the digest using a Qiagen PCR clean up spin column according to manufacturer's instructions, and the vector backbone DNA was purified by subjecting the eluate from the column to agarose gel electrophoresis to resolve the various DNA digestion products according to mass. A gel slice containing the 7.7 kBp backbone fragment was excised, and the DNA recovered from the agarose slice using the Qiagen Gel Extraction kit according to manufacturer's instructions. The concentration of DNA present in the vector preparations was determined by ethidium bromide staining in an 0.8% agarose gel following electrophoresis, by comparison to a DNA sample composed of various bands of known size and mass (High DNA Mass Ladder, Life Technologies, 10406-016). Vector preparations were quality controlled in series of test ligations as follows: vector alone control reaction, composed of x μL vector DNA (30 fmol), z μL water, 4 μL 5× ligase buffer, 1 μL T4 DNA ligase (BRL, 5 U/μL, #15224-041), where x+z=15 μL; and a vector+insert reaction, composed of x μL vector DNA (30 fmol), y μL insert DNA (90 fmol), z μL water, 4 μL 5× ligase buffer, 1 μL T4 DNA ligase, where x+y+z=15 μL. Ligation reactions were incubated at 16° C. for at least 16 hours. At the end of the incubation period, ligation products were precipitated under ethanol, the ethanol decanted and the precipitate washed three times with 70% EtOH, and the pellet dried and resuspended in 20 μL of water. One microliter of resuspended DNA solution was electrotransformed into DH10B electrocompetent cells (Life Technologies, 18290-015) according to manufacturers instructions. Following transformation, bacteria was recovered in 960 μL of room temperature SOC media, and recovery mixtures incubated at 37° C. in a rotary shaker, 250-300 rpm, for at least 40 minutes. After the recovery period, 4 ten-fold serial dilutions of each transformation culture were created, i.e., 1:10, 1:100, 1:1000, and 1:10000, and 50 μL of each bacterial dilution mixture was plated on LB-agar plates containing carbenicillin. Plates were incubated at 37° C. overnight, and scored the following morning. Stock solutions of the double-restricted vector were aliquoted and stored frozen at −20° C., preferably in 30 fmol/tube amounts.

3. Preparation of Normalized, Randomly Fragmented cDNAs from Cell Line mRNA

Total RNA was harvested from eight cell lines: A549, HCT116, HT1080-3'ss, MALME-3M, H460, MCF-7, IGROV1 and AHCN using a Qiagen RNeasy kit, according to manufacturer's instructions. RNA was normalized using driver cDNA that was covalently bound to oligtex beads (Qiagen). The driver cDNA was synthesized from 100 μg total RNA, by hybridizing the mRNA present in the total RNA to oligo(dT) covalently bound to the latex bead, then reverse transcribing the bound mRNA using the oligo(dT) as primer. RNA was eluted from the bead-bound cDNA by heat denaturation. Tester total RNA was normalized in 50 μg batches. Hybridizations were allowed to proceed for 10 min at 37° C., at which time normalized RNA was recovered from the supernatant above the beads. Multiple normalization reactions were pooled, and the mRNA extracted from the normalized RNA pool using Qiagen oligotex beads and by following manufacturer's protocols. The extent of normalization was assessed by semi-quantitative reverse-transcriptase-polymerase chain reaction (RT-PCR) analysis of transcripts for three genes (β-actin, c-myc, and E2F1), removal of an aliquot of the PCR products at 25, 30, and 35 cycles, and ethidium bromide staining of agarose gels containing the PCR products for each gene-specific RT-PCR from each reaction time point.

The normalized mRNA fractions from each of the 8 cell lines were pooled, and the pooled samples boiled for 2-5 minutes to fragment the mRNA. First strand cDNA synthesis was performed using the fragmented mRNAs as template, an Asc I-N₁₀ random primer 5′-ACC CCT CAC TTG GCG CGC CN₁₀-3′ (SEQ ID NO:21) and the Omniscript Reverse Transcriptase kit (Qiagen) following manufacturer's instructions. Second strand synthesis was performed using the method of Gubler and Hoffmnan Gene 25:263-9, 1989) using the SuperScript double strand cDNA synthesis kit (Life Technologies, #11917-010). The resultant double strand cDNAs were blunted using T4 DNA polymerase (NEB, #M0203S), then ligated to double stranded adapters, produced by annealing the oligonucleotides 5′-GGC TGG GTG TGG CGG ACC G-3′ (SEQ ID NO:22) and 5′-CGG TCC GCC ACA CCC AG-3′ (SEQ ID NO:23) to yield adapters containing an Rsr II restriction site.

4. Preparation of Inserts for Starting POLA1U Library

Normalized cDNA fragments generated as described above were subjected to 22 cycles of PCR to amplify the inserts, using primers 5′-ACC CCT CAC TTG GCG CGC C-3′ (SEQ ID NO:29) and 5′-GGC TGG GTG TGG CGG ACC G-3′ (SEQ ID NO:24). PCR products were purified using a Qiagen PCR kit following manufacturer's instructions. The concentration of PCR product in the eluate was determined by resolving the DNA present in an aliquot of the eluate by 2% agarose gel electrophoresis. The fluorescent intensity of the PCR product band was compared to the intensity of bands in a DNA sample composed of a mixture of DNA fragments of known size and mass (Low DNA Mass Ladder, Life Technologies, 10068-013). Approximately 100 ng of PCR product per reaction was digested with Asc I (NEB, #R0558S) and Rsr II restriction endonucleases for 2 hours at 37° C. DNA was recovered from the digestion using a Qiagen PCR clean up kit following manufacturer's instructions. The concentration of restricted PCR products in the eluate was determined by ethidium bromide staining as described above.

5. Isolation of GSEs for POLA1A, POLA1B, or POLA1C Libraries.

Transduced HCT116-16 cells were treated with 50 μM isopropylthio-β-galactoside (IPTG), and floating cells were collected at times 0, 24, 48, and 72 hours post-IPTG treatment and fixed with methanol. Caspase 3 positive cells were collected from the 0 and 24 hour time points by staining the fixed cells with a monoclonal antibody against caspase 3, and selecting stained cells by fluorescence activated cell sorting (FACS). Pilot experiments revealed that floating cells from 48 and 72 hour time points were greater than 95% caspase 3 positive, therefore these cells were not subjected to FACS, but instead were used en masse. Genomic DNA was isolated from the collected cells using one of two methods. For the first round of screening, genomic DNA was recovered from 2×10⁶ cells transduced with the POLA1U library, by resuspending the cells in 200 μL of PBS and using the Qiagen DNeasy kit, following manufacturer's instructions except that the DNA was purified from the proteinase K/lysis buffer by ethanol precipitation rather than using the kit columns. Pelleted genomic DNA was washed with 70% ethanol, and resuspended in 200 μL of TE.or Tris, pH 8.0 before use in PCR. For the second and third rounds of screening, after transduction of HCT116-16 cells with the POLA1A and POLA1B libraries, respectively, genomic DNA was recovered from cells using the Qiagen DNeasy kit, following manufacturer's instructions using the kit columns. Recovered genomic DNA was quantitated using the PicoGreen DNA quantitation kit (Molecular Probes) in a fluorometric assay performed according to manufacturer's instructions.

GSEs were recovered from the integrated proviruses contained in the harvested genomic DNA using PCR and the following reaction recipe: 10 μL genomic DNA solution, about 1 μg DNA, 5 μL of 3.3 μM p5x primer 5′-TCT GCA GCC CAG GTA AAA TTC GCT AGC CTC TAG T-3′ (SEQ ID NO:25), 5 μL of 3.3 μM p6x primer 5′-GAG GAA CAA GGA AAG CTG AAT ACA CGA CCC GTG AT-3′ (SEQ ID NO:26), 2 μL of 10 mM dNTP mix, 17 μL of H₂O, 10 μL 5× PCR buffer, 1 μL of Thermozyme (InVitrogen E120-01). Cycling conditions for the PCR were: 95° C. for 3 min.; followed by 30 cycles of 95° C. for 30 sec., 68° C. for 30 sec., 72° C. for 1 min.; followed by 72° C. for 10 min., followed by a soak at 4° C. At least 10, and typically 96 reactions were performed in parallel.

Two hundred μL of pooled PCR product from the genomic PCR samples was purified from proteins and salts using a Qiagen PCR clean up kit following manufacturer's instructions. The concentration of PCR product in the eluate was determined by resolving the DNA present in an aliquot of the eluate by 2% agarose gel electrophoresis and then comparing the fluorescent intensity of the PCR product band to the intensity of bands in a DNA sample composed of a mixture of DNA fragments of known size and mass (Low DNA Mass Ladder, Life Technologies, 10068-013). Multiple parallel restriction digests were then set up using samples of the purified PCR product present in the eluate using the following recipe: 10 μL 10× NEB buffer #4 (final 1× concentration: 20 mM Tris-acetate, 10 mM magnesium acetate, 50 mM potassium acetate, 1 mM dithiothreitol), 1 μL 100× BSA (NEB), 7 μL Fse I restriction endonuclease (2 U/μL, NEB #R0588S), 3 μL Rsr II restriction endonuclease (4 U/μL, NEB #R0501S), ×μL aliquot of PCR product, about 100 ng, 79 μL water, to bring total digestion volume up to 100 μL. Restriction digests were incubated at 37° C. for 3 hours. DNA products from the digest were separated from proteins and salts and concentrated using a Zymo DNA Clean & Concentrator-5 concentrator kit (#D4004), following the manufacturer's instructions with the following modifications: after addition of DNA binding buffer to each of the digestion reactions, all of the reactions were spun through the same column to concentrate 600 to 800 ng of digested insert onto a single column. Columns were washed according to the manufacturer's protocols, and the DNA eluted from the column by two sequential additions of 8 μL of 50 mM Tris-HCl, pH 8.5. DNA of desired sizes (100-500 bp) was recovered from the concentrated eluate by purification using gel electrophoresis on 1% low melting point agarose (NuSieve GTG agarose, FMC bioproducts) gels. DNA bands in the gel were visualized following ethidium bromide staining of the gel, using a hand-held shortwave ultraviolet light source. Gel slices containing the desired DNA were excised using clean razor blades, and DNA extracted from the gel slice using the Qiagen gel purification kit, following manufacturer's instructions. The concentration of restricted and purified PCR product was determined by ethidium bromide staining of an agarose gel containing an aliquot of the purified PCR product, and a DNA sample of known composition and mass, as described above

6. cDNA Library Preparation

Ligation reactions for each batch of insert prepared were set up as follows: (reaction 1) Vector control reaction: x μL vector DNA (150 ng), z μL water, 4 μL 5× ligase buffer, 1 μL T4 DNA ligase (BRL, 5 U/μL, #15224-041), where x+z=15 μL; (reaction 2) Vector+insert: x μL vector DNA (150 ng), y μL insert DNA (12 ng); z μL water; 4 μL 5× ligase buffer; 1 μL T4 DNA ligase, where x+y+z=15 μL. Ligation mixtures were incubated at 4° C. for at least 16 hours. At the end of the ligation period, ligation products were precipitated under ethanol, the ethanol decanted and the precipitate washed three times with 70% EtOH, and the pellet dried and resuspended in 20 μL of water. One μL of resuspended ligation product was used to electrotransform DH10B electrocompetent cells (Life Technologies, 18290-015) according to manufacturers instructions; the balance of the ligation mixture was stored at −20° C. Following transformation, bacteria was recovered in 960 μL of room temperature SOC media, and recovery mixtures incubated at 37° C. in a rotary shaker, 250-300 rpm, for at least 40 minutes. After the recovery period, 4 ten-fold serially diluted samples (i.e., 1:10, 1:100, 1:1000, and 1:10000) of each transformation culture were set up, and 50 μL from each dilution was plated on LB-agar plates containing carbenicillin. Plates were incubated at 37° C. overnight, and colony counts for each plate scored the following morning.

Insert sizes in a subset of 48 clones were determined by performing PCR directly on bacterial colonies as follows. A disposable pipette tip was used to harvest a single bacterial colony from the LB plate of interest. The colony was transferred into 25 μL of water, carefully swishing the tip to dislodge the bacterial colony. Five microliters of bacterial solution was spotted to an LB plate and allowed to incubate overnight. PCR was performed on the bacterial solution using the following recipe: 2 μL 10 mM primer M13F(17) 5′-GTA AAA CGA CGG CCA GT-3′ (SEQ ID NO:27), 2 μL 10 mM primer p6X 5′-TCT GCA GCC CAG GTA AAA TTC GCT AGC CTC TAG T-3′ (SEQ ID NO:28), 4 μL 10× PCR buffer, 1 μL 25 mM dNTP mix, 0.5 μL Taq DNA polymerase (Roche, 1146165), 10.5 μL PCR grade water, 20 μL of bacterial solution. The cycling parameters were 95° C. for 3 min., then 25 cycles of 95° C. for 30 sec., 60° C. for 30 sec., 72° C. for 1 min., followed by 72° C. for 5 min., and a 4° C. soak. At the completion of the PCR, 10 μL of each PCR product was resolved on a 2% agarose gel containing ethidium bromide. DNA mobility for each of the samples was evaluated. The balance of the PCR product was submitted for DNA sequencing to determine the sequence content of the inserts for these 48 clones.

Ligations described above were used for further electro-transformations. The calculated cfu/μg for each of the QC controlled ligations was used to compute the total number of electrotransformations required to achieve the required complexity for the library being constructed, e.g., POLA1U: 40 million; POLA1A: 10 million; POLA1B: 2 million; POLA1C: 1 million. Multiple electrotransformations were performed in parallel, using 1 μL of ligation mix per transformation as described above. At the end of the 40-minute recovery period following the electrotransformation, up to 10 independent transformations were pooled, and 50 μL from these pooled samples used to establish 4 ten-fold serially diluted samples (i.e., 1:10, 1:100, 1:1000, and 1:10000). Fifty μL of each serial dilution (i.e., 1:10, 1:100, 1:1000, and 1:10000) was plated on LB-agar plates containing carbenicillin. The remaining volumes of undiluted and diluted transformation solutions were used to seed a bacterial culture flask containing 0.5L of LB broth, after which the seeded flask was incubated at 30° C. overnight, about 14-16 hours, in a rotary shaker at 300 rpm. Plates from the serially diluted samples were incubated at 37° C. overnight, and colony counts for each plate scored the following morning to determine the total number of colonies seeded into the 0.5L culture. Library plasmid DNA was recovered from the 0.5L cultures using a Qiagen Maxiprep plasmid kit, according to manufacturer's instructions.

The targets of the invention are explicitly disclosed in Table 1, which contains a common name for the gene and the GENBANK accession number, which can be found at www.ncbi.gov.

TABLE 1

| Accession | Common | |
|---|---|---|
| Number | Name | Description |
| XM_050724 | APLP2 | Homo sapiens amyloid beta (A4) precursor-like protein 2 (APLP2) |
| XM_041911 | ENSA | Homo sapiens endosulfine alpha (ENSA) |
| NM_001999 | FBN2 | Homo sapiens fibrillin 2 (congenital contractural arachnodactyly) (FBN2) |
| XM_053531 | FIBL-6 | Homo sapiens fibulin 6 (FIBL-6), Mrna |
| NM_000311 | PRNP | Homo sapiens prion protein (p27-30) (Creutzfeld-Jakob disease, |
| | | Gerstmann-Strausler-Scheinker syndrome, fatal familial insomnia) (PRNP) |
| NM_013403 | STRN4 | Homo sapiens striatin, calmodulin binding protein 4 (STRN4) |
| NM_004736 | XPR1 | Homo sapiens xenotropic and polytropic retrovirus receptor (XPR1) |
| NM_021095 | SLC5A6 | Homo sapiens solute carrier family 5 (sodium-dependent vitamin |
| | | transporter), |
| | | member 6 (SLC5A6) |
| NM_001859 | SLC31A1 | Homo sapiens solute carrier family 31 (copper transporters), member 1 |
| | | (SLC31A1) |
| NM_005262 | GFER | Homo sapiens growth factor, erv1 (S. cerevisiae)-like (augmenter of liver |
| | | regeneration) |
| | | (GFER) |
| NM_006201 | PCTK1 | PCTAIRE protein kinase 1 (PCTK1), transcript variant 1 |

It should be understood that the foregoing disclosure emphasizes certain specific embodiments of the invention and that all modifications or alternatives equivalent thereto are within the spirit and scope of the invention as set forth in the appended claims.

What we claim is:

1. A method of identifying a compound that induces apoptosis in a cell, comprising:
a. contacting the cell with a putative apoptosis-inducing compound;
b. determining whether the compound inhibits a target selected from the group consisting of APLP2, ENSA, FBN2, FIBL-6, PRNP, STRN4, XPR1, SLC5A6, SLC31A1, GFER, and PCTK1.

2. The method, as claimed in claim 1, wherein the target has been validated as being involved in tumor cell growth.

3. The method, as claimed in claim 2, wherein the target has been validated as being involved in tumor cell growth by a process comprising;
a. inhibiting the target in a cell by a method selected from the group consisting of gene knock-out, anti-sense oligonucleotide expression, use of RNAi molecules and GSE expression;
b. assaying the cell for the ability of the cell to grow.

4. The method, as claimed in claim 1, wherein the cell is selected from tumor cell lines.

5. The method, as claimed in claim 1, wherein the step of determining is selected from the group consisting:
a. assaying for reduced expression of the target; and
b. assaying for reduced activity of the target.

6. The method, as claimed in claim 5, wherein the expression of the target is measured by polymerase chain reaction.

7. The method, as claimed in claim 5, wherein the expression of the target is measured using an antibody that specifically recognizes the target.

8. The method, as claimed in claim 5, wherein the activity of the target is measured by measuring the amount of a product generated in a biochemical reaction mediated by the target.

9. The method, as claimed in claim 5, wherein the activity of the target is measured by measuring the amount of a substrate consumed in a biochemical reaction mediated by the target.

10. The method, as claimed in claim 1, wherein the inhibitor is identified by:
a. determining the three-dimensional structure of the target; and
b. determining the three-dimensional structure of an inhibitor by using computer software capable of modeling the interaction of the target and putative test compounds.

11. The method, as claimed in claim 1, wherein the compound that induces apoptosis inhibits growth of tumor cells.

12. The method, as claimed in claim 1, wherein the target is APLP2.

13. The method, as claimed in claim 1, wherein the target is ENSA.

14. The method, as claimed in claim 1, wherein the target is FBN2.

15. The method, as claimed in claim 1, wherein the target is FIBL-6.

16. The method, as claimed in claim 1, wherein the target is PRNP.

17. The method, as claimed in claim 1, wherein the target is STRN4.

18. The method, as claimed in claim 1, wherein the target is XPR1.

19. The method, as claimed in claim 1, wherein the target is SLC5A6.

20. The method, as claimed in claim 1, wherein the target is SLC31A1.

21. The method, as claimed in claim 1, wherein the target is GFER.

22. The method, as claimed in claim 1, wherein the target is PCTK1.

23. A method for inducing apoptosis in a cell by inhibiting a target selected from the group consisting of APLP2, ENSA, FBN2, FIBL-6, PRNP, STRN4, XPR1, SLC5A6, SLC31A1, GFER, and PCTK1.

24. The method, as claimed in claim 23, wherein the target has been validated as being involved in tumor cell growth.

25. The method, as claimed in claim 24, wherein the target has been validated as being involved in tumor cell growth by a process comprising;
a. inhibiting the target in a cell by a method selected from the group consisting of gene knock-out, anti-sense oligonucleotide expression, use of RNAi molecules and GSE expression;
b. assaying the cell for the ability of the cell to grow.

26. The method, as claimed in claim 23, wherein the step of inhibiting is conducted by contacting a cell with an inhibitor of the target and the inhibitor induces apoptosis in the cell.

27. The method, as claimed in claim 23, wherein the target is APLP2.

28. The method, as claimed in claim 23, wherein the target is ENSA.

29. The method, as claimed in claim 23, wherein the target is FBN2.

30. The method, as claimed in claim 23, wherein the target is FIBL-6.

31. The method, as claimed in claim 23, wherein the target is PRNP.

32. The method, as claimed in claim 23, wherein the target is STRN4.

33. The method, as claimed in claim 23, wherein the target is XPR1.

34. The method, as claimed in claim 23, wherein the target is SLC5A6.

35. The method, as claimed in claim 23, wherein the target is SLC31A1.

36. The method, as claimed in claim 23, wherein the target is GFER.

37. The method, as claimed in claim 23, wherein the target is PCTK1.

38. A method for the diagnosis of a tumor comprising determining the level of a marker in a patient sample, the level of the marker being indicative of the presence of tumor cells, wherein the marker is selected from the group consisting of APLP2, ENSA, FBN2, FIBL-6, PRNP, STRN4, XPR1, SLC5A6, SLC31A1, GFER, and PCTK1.

39. The method as claimed in claim 38, wherein the level of the marker is determined by identifying the marker as a cell surface molecule in tissue.

40. The method as claimed in claim 38, wherein the level of the marker is determined by detecting the marker in soluble form in a bodily fluid.

41. The method as claimed in claim 40, wherein the bodily fluid is serum.

42. The method as claimed in claim 40, wherein the marker level is determined by contacting a patient sample with an antibody, or a fragment thereof, that binds specifically to the marker and determining whether the anti-marker antibody or fragment thereof has bound to the marker.

43. The method as claimed in claim 40, wherein the marker level is determined using a first monoclonal antibody that binds specifically to the marker and a second antibody that binds to the first antibody.

44. The method as claimed in claim 40, wherein the bodily fluid is immobilized.

45. The method as claimed in claim 38, wherein the method is used to determine the prognosis for cancer in the patient.

46. The method as claimed in claim 38, wherein the method is used to determine the susceptibility of the patient to a therapeutic treatment.

47. The method, as claimed in claim 38, wherein the target is APLP2.

48. The method, as claimed in claim 38, wherein the target is ENSA.

49. The method, as claimed in claim 38, wherein the target is FBN2.

50. The method, as claimed in claim 38, wherein the target is FIBL-6.

51. The method, as claimed in claim 38, wherein the target is PRNP.

52. The method, as claimed in claim 38, wherein the target is STRN4.

53. The method, as claimed in claim 38, wherein the target is XPR1.

54. The method, as claimed in claim 38, wherein the target is SLC5A6.

55. The method, as claimed in claim 38, wherein the target is SLC31A1.

56. The method, as claimed in claim 38, wherein the target is GFER.

57. The method, as claimed in claim 38, wherein the target is PCTK1.


SEQUENCE LISTING (33 sequences)

SEQ ID NO: 1 - DNA, 27 bp, Artificial Sequence
  PCR primer
accatggcctcaccgttgacccgcttt

SEQ ID NO: 2 - DNA, 25 bp, Artificial Sequence
  primer
ctagcggctgtggtagcagatgaga

SEQ ID NO: 3 - DNA, 29 bp, Artificial Sequence
  primer
ctacggatccaccatggcctcaccgttga

SEQ ID NO: 4 - DNA, 35 bp, Artificial Sequence
  primer
gtacatcgatctagcggctgtggtagcagatgaga

SEQ ID NO: 5 - DNA, 61 bp, Artificial Sequence
  primer
agctgtaaaacgacggccagtgagcgtttaaacgaattccagactagtggccggccgtgca

SEQ ID NO: 6 - DNA, 53 bp, Artificial Sequence
  multiple cloning site
cggccggccactagtctggaattcgtttaaacgctcactggccgtcgttttac

SEQ ID NO: 7 - DNA, 32 bp, Artificial Sequence
  multiple cloning site
aattctgcagcccaggtaaaattcgctagcct

SEQ ID NO: 8 - DNA, 32 bp, Artificial Sequence
  multiple cloning site
ctagaggctagcgaattttacctgggctgcag

SEQ ID NO: 9 - DNA, 72 bp, Artificial Sequence
  vector insert
cggtccgtgagtgagtgaggcgcgccggatcctaacctaggtaatcatggtcatagctgtttcctgcagggc

SEQ ID NO: 10 - DNA, 80 bp, Artificial Sequence
  vector insert
ggccgccctgcaggaaacagctatgaccatgattacctaggttaggatccggcgcgcctcactcactcacggaccgtgca

SEQ ID NO: 11 - DNA, 35 bp, Artificial Sequence
  vector insert
gatcccgggtcgtgtattcagctttccttgttcct

SEQ ID NO: 12 - DNA, 35 bp, Artificial Sequence
  vector insert
ctagaggaacaaggaaagctgaatacacgacccgg

SEQ ID NO: 13 - DNA, 43 bp, Artificial Sequence
  primer
catcaagcttggccggccacgcgtgttggtaaaatggaagacg

SEQ ID NO: 14 - DNA, 35 bp, Artificial Sequence
  primer
cacgtggatatcttacaatttggactttccgccct

SEQ ID NO: 15 - DNA, 31 bp, Artificial Sequence
  primer
ttgtaagatatccacgtgttgacaattaatc

SEQ ID NO: 16 - DNA, 45 bp, Artificial Sequence
  primer
catcagatctgtcgaccggaccgacgcgtccacgaagtgcttagc

SEQ ID NO: 17 - DNA, 43 bp, Artificial Sequence; identical to SEQ ID NO: 13
  primer

SEQ ID NO: 18 - DNA, 45 bp, Artificial Sequence; identical to SEQ ID NO: 16
  primer

SEQ ID NO: 19 - DNA, 43 bp, Artificial Sequence; identical to SEQ ID NO: 13
  primer

SEQ ID NO: 20 - DNA, 33 bp, Artificial Sequence
  primer
catcgtcgaccggaccgccacgaagtgcttagc

SEQ ID NO: 21 - DNA, 29 bp, Artificial Sequence
  primer
acccctcacttggcgcgccnnnnnnnnnn

SEQ ID NO: 22 - DNA, 19 bp, Artificial Sequence
  linker
ggctgggtgtggcggaccg

SEQ ID NO: 23 - DNA, 17 bp, Artificial Sequence
  linker
cggtccgccacacccag

SEQ ID NO: 24 - DNA, 19 bp, Artificial Sequence
  primer
ggctgggtgtggcggaccg

SEQ ID NO: 25 - DNA, 34 bp, Artificial Sequence
  primer
tctgcagcccaggtaaaattcgctagcctctagt

SEQ ID NO: 26 - DNA, 35 bp, Artificial Sequence
  primer
gaggaacaaggaaagctgaatacacgacccgtgat

SEQ ID NO: 27 - DNA, 17 bp, Artificial Sequence
  primer
gtaaaacgacggccagt

SEQ ID NO: 28 - DNA, 34 bp, Artificial Sequence; identical to SEQ ID NO: 25
  primer

SEQ ID NO: 29 - DNA, 19 bp, Artificial Sequence
  primer
acccctcacttggcgcgcc

SEQ ID NO: 30 - DNA, 29 bp, Artificial Sequence
  primer
gtaaaacgacggccagtgagcgtttaaac

SEQ ID NO: 31 - DNA, 29 bp, Artificial Sequence
  primer
ggaaacagctatgaccatgattacctagg

SEQ ID NO: 32 - DNA, 25 bp, Artificial Sequence
  primer
cagcccaggtaaaattcgctagcct

SEQ ID NO: 33 - DNA, 28 bp, Artificial Sequence
  primer
gaggaacaaggaaagctgaatacacgac